(19) United States
(12) Patent Application Publication
(10) Pub. No.: US 2025/0186649 A1
(43) Pub. Date: Jun. 12, 2025

(54) ULTRAVIOLET STERILIZATION DEVICE

(71) Applicant: XUZHOU LIYU ADVANCED TECHNOLOGY CO., LTD., Jiangsu (CN)

(72) Inventors: Guangjin WANG, Jiangsu (CN); Yitao LIAO, Jiangsu (CN)

(21) Appl. No.: 18/954,501

(22) Filed: Nov. 20, 2024

(30) Foreign Application Priority Data
Dec. 11, 2023 (CN) 202323361543.X
Jan. 12, 2024 (CN) 202410047600.7

(51) Int. Cl. A61L 9/20

(52) U.S. Cl. CPC A61L 9/20; A61L 2209/12

(57) ABSTRACT

The application relates to an ultraviolet sterilization device comprising: a chamber; an inlet in fluid communication with one end of the chamber; an outlet in fluid communication with the other end of the chamber; a fan assembly arranged at the inlet and configured to operate to form an airflow that flows from the inlet into the chamber and then flows out through the outlet; a light concentrating reflector arranged in the chamber near the outlet, the light concentrating reflector having a periphery hindering the airflow from flowing from the chamber to the outlet, and the light concentrating reflector comprising a groove wall forming a reflective cavity confronting a flow direction of the airflow in the chamber; and a light emitter arranged in the reflective cavity such that at least part of light emitted by the light emitter is reflected by the groove wall to be parallel to the flow direction.

### TECHNICAL FIELD

The present application relates to the field of sterilization technology, and in particular to an ultraviolet sterilization device.

### BACKGROUND

Currently, most ultraviolet (UV) sterilization systems use high-pressure mercury lamps containing heavy metals. These systems lack dedicated ventilation ducts, light path designs and cooling systems. The absence of these features significantly weakens the sterilization effect. For instance, non-concentrated and non-sealed airflow paths lead to reduced air circulation, diminishing the sterilization space. Without the light path designs, the propagation of light is subject to absorption, scattering, and multiple reflections, with over 30% of light energy lost to these phenomena, directly reducing sterilization efficiency.

### SUMMARY

In view of this, the present application provides an ultraviolet sterilization device to solve a problem that ultraviolet light from the ultraviolet sterilization device can be easily scattered and absorbed, leading to insufficient contact of the ultraviolet light with air to be sterilized and inadequate sterilization efficiency.

In an aspect of the present application, there is provided an ultraviolet sterilization device, comprising: a chamber; an inlet in fluid communication with one end of the chamber; an outlet in fluid communication with the other end of the chamber; a fan assembly arranged at the inlet, the fan assembly being configured to operate to form an airflow that flows from the inlet into the chamber and then flows out through the outlet; a light concentrating reflector arranged in the chamber near the outlet, the light concentrating reflector having a periphery hindering the airflow from flowing from the chamber to the outlet, and the light concentrating reflector comprising a groove wall forming a reflective cavity confronting a flow direction of the airflow in the chamber; and a light emitter arranged in the reflective cavity such that at least part of light emitted by the light emitter is reflected by the groove wall to be parallel to the flow direction of the airflow in the chamber.

Optionally, the groove wall forming the reflective cavity is provided with a parabolic concentrating reflective surface layer to reflect the at least part of the light emitted by the light emitter to be parallel to the flow direction of the airflow in the chamber.

Optionally, a heat dissipation fin is provided at a side of the light concentrating reflector that is faced away from the reflective cavity.

Optionally, the ultraviolet sterilization device has a first inner wall forming the chamber and a second inner wall forming the outlet, the first inner wall being provided with a first reflective layer, and the second inner wall facing a side of the light concentrating reflector that is faced away from the reflective cavity and the second inner wall being provided with a light absorbing layer.

Optionally, the inlet and the outlet are arranged at two ends of the chamber respectively.

Optionally, an air intake plate with a plurality of air holes is provided at the inlet and an air outlet plate with a plurality of air holes is provided at the outlet.

Optionally, the fan assembly has fan blades with a rotation axis that is parallel to a center line of each of the plurality of air holes of the air intake plate.

Optionally, the fan assembly has fan blades with a fourth reflective layer configured to reflect light from the chamber back into the chamber so as to prevent light leakage.

Optionally, the light emitter comprises a light-emitting semiconductor chip.

Optionally, a mounting slot for mounting the light emitter is provided at the bottom of the groove wall forming the reflective cavity and a thermal conductive adhesive is provided for bonding the light emitter with the mounting slot.

Optionally, the light concentrating reflector is detachably connected to the heat dissipation fin.

Optionally, the second inner is provided with an air guide surface inclined relative to the air outlet plate so as to guide the airflow to flow out through the plurality of air holes of the air outlet plate.

Optionally, the light emitter is an ultraviolet LED light strip.

Optionally, the ultraviolet light strip is a UVC-LED light strip.

The present application provides the ultraviolet sterilization device with the light emitter, the fan assembly and the light concentrating reflector in the chamber. The fan assembly is provided at the inlet such that the fan assembly sucks in air with bacteria through the inlet to form an airflow, and then the airflow enters the chamber with a certain space and reflection performance. The reflective cavity of the light concentrating reflector confronts the flow direction of the airflow in the chamber such that the airflow is sterilized by the light emitted by the light emitter and then reflected and concentrated by the light concentrating reflector, and then the sterilized airflow, as clean air, is discharged from the outlet. As the light emitted by the light emitter is reflected by the light concentrating reflector in a direction parallel to the flow direction of the airflow, an efficient sterilization of the air circulating through the ultraviolet sterilization device is achieved. Additionally, the circulating air rapidly dissipates heat from the light concentrating reflector, reducing an operating temperature of the light emitter and extending its lifespan.

### BRIEF DESCRIPTION OF THE DRAWINGS

To clearly illustrate the technical solutions of the embodiments in this application, a simple introduction to the accompanying drawings of the embodiments is provided below. It is evident that the drawings mentioned here represent only some embodiments of this application and are not intended as a limitation.

FIG. 1 is a schematic view of an ultraviolet sterilization device provided in an embodiment of this application.

FIG. 2 is a bottom schematic view of the ultraviolet sterilization device in FIG. 1.

FIG. 3 is a cross-sectional schematic view of the ultraviolet sterilization device in FIG. 1.

FIG. 4 is an enlarged schematic view of part A in FIG. 3.

FIG. 5 is a schematic diagram of a light path of the ultraviolet sterilization device in FIG. 1.

FIG. 6 is a schematic diagram of a light concentrating reflector and a light emitter of the ultraviolet sterilization device in FIG. 1.

### REFERENCE NUMBER LIST

101. front frame; 102. rear shell; 103. first inner wall; 104. second inner wall; 24. mounting bracket; 1. chamber; 11. inlet; 111. air intake plate; 12. outlet; 121. air outlet plate; 13. fan assembly; 2. light concentrating reflector; 21. light emitter; 22. reflective cavity; 221. groove wall; 222. mounting slot; 223. thermal conductive adhesive; 23. heat dissipation fin.

### DETAILED DESCRIPTION OF EMBODIMENTS

The technical solutions in the embodiments of this application are described clearly and completely in conjunction with the accompanying drawings. Obviously, the described embodiments are only a part of the embodiments of this application, not all of them. Based on the embodiments in this application, all other embodiments obtained by those skilled in the art without creative work fall within the protection scope of this application.

In the description of this application, it should be understood that terms like “first,” “second” are used only for descriptive purposes and should not be taken to indicate or imply relative importance or implicitly specify the quantity of the indicated technical features. Thus, features defined with “first,” “second” may include one or more of such features. In the description of this application, the term “multiple” means two or more, unless specifically defined otherwise.

In this application, the term “exemplary” is used to mean “serving as an example, instance, or illustration.” Any embodiment described as “exemplary” in this application is not necessarily to be construed as preferred or advantageous over other embodiments. The description provided here is to enable any person skilled in the art to implement and use this application. For explanatory purposes, details are provided in the following description. It should be understood that the skilled in the art can realize this application without using these specific details. In other instances, well-known structures and processes are not described in detail to avoid unnecessarily obscuring the description of this application. Therefore, this application is not intended to be limited to the embodiments shown, but is to be accorded the broadest scope consistent with the principles disclosed herein.

In the related art, an ultraviolet sterilization device usually has an ultraviolet sterilization lamp in a chamber for sterilization and the ultraviolet sterilization lamp is generally placed in the chamber. Such devices, due to the absence of a light concentrator for the ultraviolet sterilization lamp, do not achieve a good sterilization effect. Additionally, the ultraviolet sterilization lamp is usually set perpendicular to a flow direction of an airflow in the chamber, leading to insufficient contact of light emitted by the ultraviolet sterilization lamp with air to be sterilized and diminishing the sterilization effect. Moreover, the ultraviolet sterilization lamp used in the related art is a mercury lamp, which pose a risk of heavy metal leakage, harmful to human health.

The ultraviolet sterilization device in the embodiments of this application can be set up in biochemical laboratories where air cleanliness is highly required or used in everyday living and working environments. It can be used independently or integrated into an air conditioner or other air exchange devices.

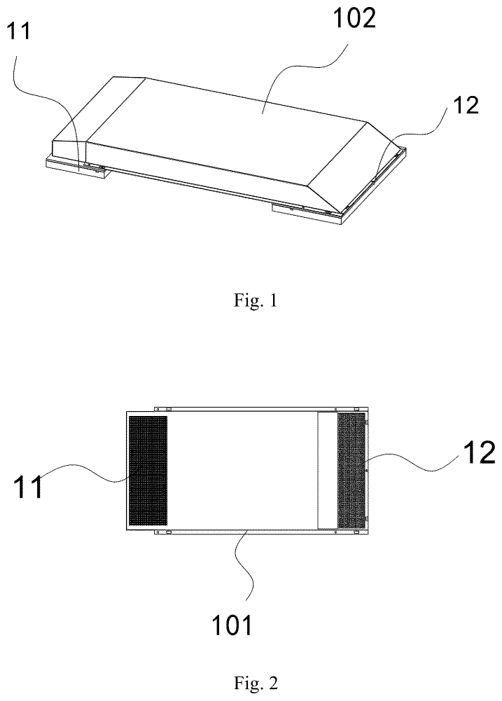

An embodiment of the present application provides an ultraviolet sterilization device, as shown in FIGS. 1 to 3, comprising a front frame 101 and a rear shell 102, the front frame 101 and the rear shell 102 together define a chamber 1, an inlet 11 and an outlet 12. The chamber 1 is in fluid communication with the inlet 11 at one end, and the chamber 1 is in fluid communication with the outlet 12 at the other end, that is, the chamber 1 is located between the inlet 11 and the outlet 12. A fan assembly is provided at the inlet 11, so as to form an airflow flowing in from the inlet 11, passing through the chamber 1 and flowing out through the outlet 12. As shown in FIG. 4, a light concentrating reflector 2 is arranged in the chamber 1, and the light concentrating reflector 2 comprises an upwind side forming a reflective cavity 22 and a leeward side. A light emitter 21 is arranged in the reflective cavity 22. A ventilation gap is formed around the light concentrating reflector 2, that is, the light concentrating reflector 2 has a periphery for forming the ventilation gap with a first inner wall 103 of the front frame 101 and the rear shell 102. The first inner wall 103 forms the chamber 1. The reflective cavity 22 confronts a flow direction of the airflow in the chamber 1.

The fan assembly 13 may comprise one fan, one column of fans and multiple rows of fans, or multiple columns and rows of fans.

The light concentrating reflector 2 is arranged close to the outlet 12, and the fan assembly 13 is arranged close to the inlet 11. The fan assembly 13 is operated to suck in air carrying bacteria to form the airflow from the inlet 11 to the chamber 1 and then to the outlet 12.

A light emitter 21 is arranged in the light concentrating reflector 2, and the light concentrating reflector 2 concentrates and reflects most of the light (for example, ultraviolet light) emitted by the light emitter 21 toward the flow direction of the airflow in the chamber 1 to sterilize the oncoming airflow.

The light concentrating reflector 2 is connected to the front frame 101 and/or the rear shell 102 via a mounting bracket 24 to ensure that the light concentrating reflector 2 is facing the flow direction of the airflow. The mounting bracket can be such as a metal frame that is made of a material with a good heat dissipation performance.

A ventilation gap is formed around the light concentrating reflector 2 to allow the airflow to circulate from the inlet 11 through the chamber 1, especially the ventilation gap around the light concentrating reflector 2, to the outlet 12. The ventilation gap need to be as small as possible so as to hinder the airflow to flow from the chamber 1 to the outlet 12, while ensuring the fluid communication of the chamber 1 to the outlet 12. Thus, a sufficient contact between the air carrying the bacteria and the ultraviolet light reflected by the light concentrating reflector 2 is ensured.

The light concentrating reflector 2 can be made of a metal material, such as aluminum, with a good heat dissipation performance.

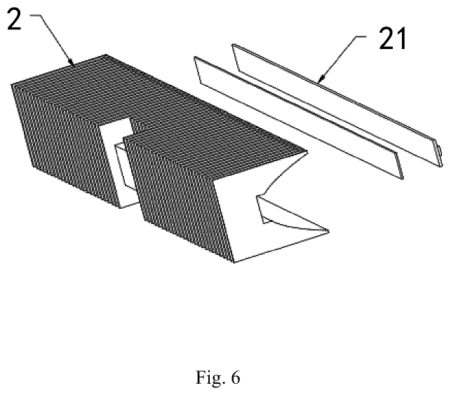

As shown in FIG. 5, most of the light emitted by the light emitter 21 can be reflected and concentrated by the light concentrating reflector 2 and can follow a light path in the chamber 1 that is mostly parallel to the direction of the airflow, that is, the light has a concentrated intensity and an enhanced light energy with a small angle. A small part of the light is reflected to the first inner wall 103 to be further reflected or absorbed.

The present application provides the ultraviolet sterilization device with the light emitter 21, the fan assembly 13 and the light concentrating reflector 2 in the chamber 1. The fan assembly 13 is provided at the inlet 11 such that the fan assembly 13 sucks in air with the bacteria through the inlet 11 to form an airflow, and then the airflow enters the chamber 1 with a certain space and reflection performance. The reflective cavity of the light concentrating reflector 2 confronts the flow direction of the airflow in the chamber 1 such that the airflow is sterilized by the light emitted by the light emitter 21 and then reflected and concentrated by the light concentrating reflector 2, and then the sterilized airflow as clean air, is discharged from the outlet 12. As the light emitted by the light emitter 21 is reflected by the light concentrating reflector 2 in a direction parallel to the flow direction of the airflow, an efficient sterilization of the air circulating through the ultraviolet sterilization device is achieved. Additionally, the circulating air rapidly dissipates heat from the light concentrating reflector 2, reducing an operating temperature of the light emitter 21 and extending its lifespan.

In an embodiment of the present application, referring to FIG. 4, a groove wall 221 forming the reflective cavity 22 of the light concentrating reflector 2 is provided with a parabolic concentrating reflective surface layer which is used to reflect a part of the light emitted by the light emitter 21 to be parallel to the flow direction of the airflow.

The light emitted by the light emitter 21 is ultraviolet light, which is used to sterilize the flowing air containing the bacteria and germs so as to form clean air.

The reflective cavity 22 is oriented to face the flow direction of the airflow in the chamber 1 such that the light emitted by the light emitter 21 in the chamber 1 has a direction that can be nearly parallel to, rather than perpendicular to the flow direction of the airflow inside the chamber 1. In this way, a contact area between the light emitter 21 in the chamber 1 and the flowing air containing the bacteria or the germs can be greatly increased, thereby greatly improving the ultraviolet sterilization device's performance.

When the light concentrating reflector 2 has a larger footprint relative to the chamber 1, the light concentrating reflector 2 would have a larger reflective surface and the chamber 1 would have a less amount of fluid communication, thereby enhancing the sterilization effect. Therefore, it is desirable to enlarge the area of the concentrating reflective surface layer of the groove wall 221 forming the reflective cavity 22 and reduce the volume of the light emitter 21 (but a certain light intensity needs to be guaranteed). While the chamber 1 has an enough amount of fluid communication and most of the light emitted by the light emitter 21 and reflected and concentrated by the light concentrating reflector 2 is parallel to the flow direction of the airflow in the chamber 1, a desirable ultraviolet sterilization effect is realized. Alternatively, the chamber 1 can be set as a rectangular parallelepiped, and the groove wall 221 can be formed as a conical groove wall, which is used to reflect a part of the light emitted by the light emitter 21, such as an ultraviolet point light source (a single ultraviolet LED), to be parallel to the flow direction of the airflow.

Referring to FIG. 4, a heat dissipation fin 23 is provided on a side of the light concentrating reflector 2 facing away from the reflective cavity 22.

Since the light emitter 21, especially a light-emitting semiconductor chip for manufacturing the ultraviolet LED, is prone to generate heat during operation, the light emitter 21 is installed at the bottom of the reflective cavity 22 and the heat dissipation fin 23 are arranged at the bottom of the light concentrating reflector 2, such that heat generated by the light emitter 21 during operation can be more easily taken away by the heat dissipation fin 23. In this way, the light emitter 21 can have a good luminous efficiency and a long service life, thereby improving the service life of the entire ultraviolet sterilization device.

Furthermore, the heat dissipation fin 23 may be made of a metal material with a good heat dissipation performance.

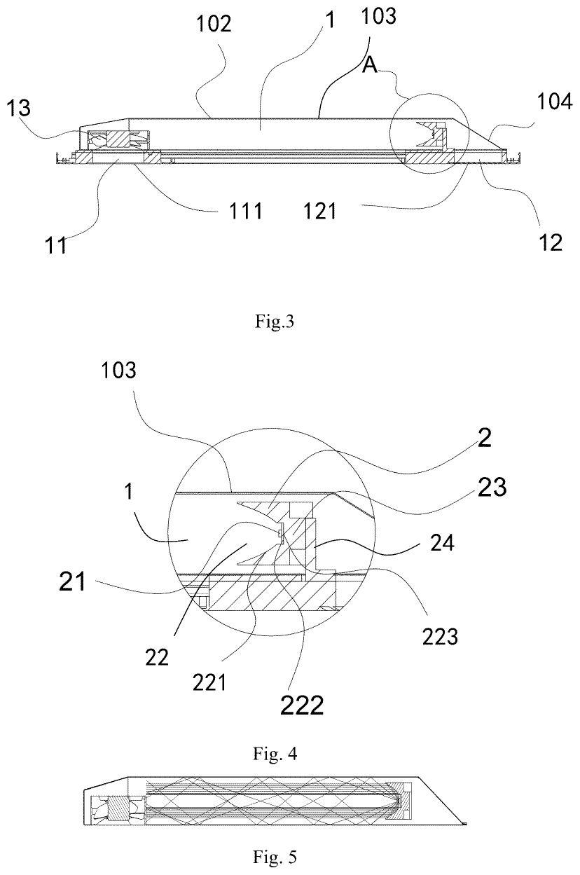

As shown in FIGS. 3 and 4, the first inner wall 103 is provided with a first reflective layer, and the front frame 101 and/or the rear shell 102 has a second inner wall 104 provided with a light absorbing layer. The second inner wall 104 faces a side of the light concentrating reflector 2 facing away from the reflective cavity 22 and forms the outlet 12.

The first reflective layer can be a coating or a deposited layer formed by thin film deposition. The first reflective layer can be a metal layer or a mirror layer. The first reflective layer in the present application uses an aluminum layer. The light-absorbing layer can be a material, such as plastic, with a good light-absorbing performance. Plastic has a good ultraviolet light-absorbing performance.

The first inner wall 103 of the front frame 101 and/or the rear shell 102 is provided with the first reflective layer to enable a part of the light emitted by the light emitter 21 into the chamber 1 to be reflected (instead of being absorbed) by the first reflective layer on the first inner wall 103, such that the part of the light can be in contact with more airflow, thereby further improving the ultraviolet sterilization effect.

Since part of the light (for example, the ultraviolet light) emitted by the light emitter 21 may be reflected by the first inner wall 103 to the second inner wall 104 through the ventilation gap around the periphery of the light concentrating reflector 2, it is necessary to provide the light-absorbing layer on the second inner wall 104 to absorb the ultraviolet light reflected to the second inner wall 104, thereby reducing a proportion of the ultraviolet light being reflected by the second inner wall 104 and leaking through the outlet 12, thereby improving a safety performance of the ultraviolet sterilization device.

As shown in FIG. 3, the inlet 11 and the outlet 12 are disposed at two ends of the chamber 1 respectively.

As shown in FIGS. 2 and 3, an air intake plate 111 with a plurality of air holes is provided at the inlet 11, and an air outlet plate 121 with a plurality of air holes is provided at the outlet 12.

Side walls disposed at two ends of the rear shell 102 form at least part of the inlet 11 and the outlet 12. The side walls enable a part of the light coming from the chamber 1 to be reflected back into the chamber 1, thereby making it difficult for the light to leak from the inlet 11 and the outlet 12 and further improving the ultraviolet sterilization effect.

As shown in FIG. 3, the fan assembly 13 is disposed inside the inlet 11, and the fan assembly 13 has fan blades with a rotation axis that is parallel to a center line of each of the plurality of air holes of the air intake plate 111, such that the fan assembly 13 can deliver all the airflow from the inlet 11 into the chamber 1.

As shown in FIG. 3, a fourth reflective layer is provided on the fan assembly 13, for example, on a side of each of the fan blades that guides the airflow into the chamber 1. The fourth reflective layer is configured to reflect the light (for example, the ultraviolet light) from the chamber 1 back into the chamber 1 to prevent light leakage.

The side wall of the rear shell 102 forming the at least part of the inlet 11 functions to reflect the ultraviolet light coming from the chamber 1 back into the chamber 1 together with the fourth reflective layer, thereby enhancing the effect of preventing light leakage and improving the ultraviolet light efficiency. Moreover, the fan blades on the fan assembly 13 have a certain inclination compared to a plane perpendicular to the rotation axis of the fan blades of the fan assembly 13, as the fan blades of the fan assembly 13 rotate, the fourth reflective layer can disrupt a reflected light path of the ultraviolet light, thereby resulting in a more uniform ultraviolet light distribution in the chamber 1.

The fourth reflective layer is provided on the fan blades, that is, on the side of each of the fan blades that guides the airflow into the chamber 1, such that the fan assembly 13 can reflect the ultraviolet light, thereby enhancing the effect of preventing ultraviolet light leakage from the inlet 11.

The light emitter 21 comprises a light emitting semiconductor chip. As shown in FIG. 6, the light emitting device 21 in the light concentrating reflector 2 can be an ultraviolet LED light strip. For example, the ultraviolet LED light strip is a UVC-LED light strip.

More than 95% of the ultraviolet sterilization devices in the art are using ultraviolet mercury lamps. The ultraviolet mercury lamps operate with a wavelength band that is relatively wide and if the ultraviolet mercury lamps operate with a wavelength below 200 nm, ozone with a pungent smell will be produced. Some human organs, such as a cornea and a respiratory mucosa, with immune functions, are going to be destroyed in a high-concentration ozone environment. In addition to containing heavy metals, the ultraviolet mercury lamps are large in size, high in power consumption, long in response time, and short in life, and need to be used with a timer under an unmanned condition.

The light emitter 21 provided as the ultraviolet LED light strip, such as the UVC-LED light strip, has a high efficiency, is safe and environmentally friendly compared to other kinds of light emitters. In addition, the airflow generated by the fan assembly 13 takes away the heat generated by the ultraviolet LED light strip. Specifically, when the ultraviolet LED light strip is in a working state, the heat emitted by the ultraviolet LED light strip can be taken away by the airflow, thereby improving the service life of the ultraviolet LED light strip.

As shown in FIG. 4, a mounting slot 222 for mounting the light emitter 21 is provided at the bottom of the groove wall 221 and a thermal conductive adhesive 223 is provided for bonding the light emitter 21 with the mounting slot 222.

The mounting slot 222 is provided with heat dissipation holes penetrating the concentrating reflector 2 for transferring the airflow from one side of the concentrating reflector 2 to the other side of the concentrating reflector 2 to pass through the heat dissipation fin 23, thereby achieving a better heat dissipation effect for the concentrating reflector 2.

It is fast to attach the light emitter 21 to the mounting slot 222 with thermal conductive adhesive 223. The thermal conductive adhesive 223 can quickly transfer the heat from the light emitter 21 to the light concentrating reflector 2, thereby facilitating a rapid heat dissipation of the light emitter 21.

The mounting slot 222 for mounting the light emitter 21 is formed at the bottom of the groove wall 221. The light emitter 21 and the mounting slot 222 are connected by a thermal conductive adhesive 223. The heat generated by the light emitter 21 can be quickly absorbed by the thermal conductive adhesive 223 and dissipated through the mounting slot 222 and the heat dissipation fin 23, thereby improving the light emitter 21's heat dissipation speed.

The light concentrating reflector 2 is detachably connected to the heat dissipation fin 23.

The detachable connection can be realized by at least one of a thermal conductive glue and a bolt. The thermal conductive glue is to enhance a thermal conductivity of the heat dissipation fin 23 and the bolt is to enhance the detachable connection's strength.

The light concentrating reflector 2 is detachably connected to the heat dissipation fin 23, such that a mold making cost of the light concentrating reflector 2 can be lowered, thereby ensuring the heat dissipation effect of the light concentrating reflector 2 especially at the back and reducing the mold making cost of the light concentrating reflector 2.

Referring to FIG. 3, the second inner wall 104 is provided with an air guide surface, which is used to guide the airflow to the plurality of air holes of the air outlet plate 121, thereby facilitating the airflow to flow smoothly.

The air guide surface can be a slope or a curved surface. The light absorbing layer is located on the air guide surface, such that when the ultraviolet light passes through the light concentrating reflector 2 and is projected onto the air guide surface, the ultraviolet light can be absorbed instead of being reflected, thereby preventing the air guide surface from reflecting the ultraviolet light to the plurality of air holes of the air outlet plate 121 and preventing the ultraviolet light leaking from the plurality of air holes of the air outlet plate 121.

The present application provides the air guide surface such that the airflow ultraviolet sterilized light can be more easily guided to the outlet 12, thereby facilitating the airflow to flow smoothly in the chamber 1.

While the basic concepts have been described above, it should be apparent to those skilled in the art that the detailed disclosure is merely illustrative and not limiting of the application. Although not explicitly described here, various modifications, improvements, and corrections to this application by those skilled in the art are within the spirit and scope of the exemplary embodiments suggested in this application.

The use of specific terminology in describing embodiments of this application, such as “an embodiment,” “one embodiment,” and/or “some embodiments,” indicates that certain features, structures, or characteristics associated with at least one embodiment of the application. Thus, it should be emphasized that the term “one embodiment” or “an embodiment” mentioned at different locations in the description may not necessarily refer to the same embodiment. Furthermore, some features, structures, or characteristics of one or more embodiments of the application may be suitably combined.

In the same vein, to simplify the disclosure of the application and aid in understanding one or more embodiments, certain features have been grouped together in a single embodiment, figure, or description thereof in the preceding text. However, such method of disclosure should not be interpreted as indicating that the claimed application requires more features than those expressly mentioned in the claims. In fact, fewer features may be required than those disclosed in a single such embodiment.

Every patent, patent application, published patent application, and other materials, like articles, books, manuals, publications, documents, etc., referenced in this application are hereby incorporated by reference in their entirety, except where they conflict with the history of this application or limit the broadest scope of this application as claimed, whether currently or later added to this application. It should be noted that if there is any inconsistency or conflict in the use of descriptions, definitions, and/or terms between the auxiliary materials of this application and the content of this application, the usage in the application itself will prevail.

1. An ultraviolet sterilization device, comprising:
a chamber;
an inlet in fluid communication with one end of the chamber;
an outlet in fluid communication with the other end of the chamber;
a fan assembly arranged at the inlet, the fan assembly being configured to operate to form an airflow that flows from the inlet into the chamber and then flows out through the outlet;
a light concentrating reflector arranged in the chamber near the outlet, the light concentrating reflector having a periphery hindering the airflow from flowing from the chamber to the outlet, and the light concentrating reflector comprising a groove wall forming a reflective cavity confronting a flow direction of the airflow in the chamber; and
a light emitter arranged in the reflective cavity such that at least part of light emitted by the light emitter is reflected by the groove wall to be parallel to the flow direction of the airflow in the chamber.

2. The ultraviolet sterilization device according to claim 1, wherein the groove wall forming the reflective cavity is provided with a parabolic concentrating reflective surface layer to reflect the at least part of the light emitted by the light emitter to be parallel to the flow direction of the airflow in the chamber.

3. The ultraviolet sterilization device according to claim 2, wherein a heat dissipation fin is provided at a side of the light concentrating reflector that is faced away from the reflective cavity.

4. The ultraviolet sterilization device according to claim 1, wherein the ultraviolet sterilization device has a first inner wall forming the chamber and a second inner wall forming the outlet, the first inner wall being provided with a first reflective layer, and the second inner wall facing a side of the light concentrating reflector that is faced away from the reflective cavity and the second inner wall being provided with a light absorbing layer.

5. The ultraviolet sterilization device according to claim 4, wherein the inlet and the outlet are arranged at two ends of the chamber respectively.

6. The ultraviolet sterilization device according to claim 5, wherein an air intake plate with a plurality of air holes is provided at the inlet and an air outlet plate with a plurality of air holes is provided at the outlet.

7. The ultraviolet sterilization device according to claim 6, wherein the fan assembly has fan blades with a rotation axis that is parallel to a center line of each of the plurality of air holes of the air intake plate.

8. The ultraviolet sterilization device according to claim 1, wherein the fan assembly has fan blades with a fourth reflective layer configured to reflect light from the chamber back into the chamber so as to prevent light leakage.

9. The ultraviolet sterilization device according to claim 1, wherein the light emitter comprises a light-emitting semiconductor chip.

10. The ultraviolet sterilization device according to claim 1, wherein a mounting slot for mounting the light emitter is provided at the bottom of the groove wall forming the reflective cavity and a thermal conductive adhesive is provided for bonding the light emitter with the mounting slot.

11. The ultraviolet sterilization device according to claim 3, wherein the light concentrating reflector is detachably connected to the heat dissipation fin.

12. The ultraviolet sterilization device according to claim 6, wherein the second inner is provided with an air guide surface inclined relative to the air outlet plate so as to guide the airflow to flow out through the plurality of air holes of the air outlet plate.

13. The ultraviolet sterilization device according to claim 9, wherein the light emitter is an ultraviolet LED light strip.

14. The ultraviolet sterilization device according to claim 13, wherein the ultraviolet light strip is a UVC-LED light strip.
